(19) United States
(12) Patent Application Publication
(10) Pub. No.: US 2023/0203420 A1
(43) Pub. Date: Jun. 29, 2023

(54) METHOD FOR PRESSURIZING CELLS GROWN IN HYDROGEL TO INDUCE HYPERTROPHY

(71) Applicant: Upside Foods, Inc., Berkeley, CA (US)

(72) Inventors: Matthew Leung, Richmond, CA (US); Morgan Laurence Rease, Emmeryville, CA (US); David Kim, Berkeley, CA (US); Jeanne Li, Berkeley, CA (US); Emily Quan, Berkeley, CA (US)

(21) Appl. No.: 18/050,237

(22) Filed: Oct. 27, 2022

Related U.S. Application Data
(60) Provisional application No. 63/294,700, filed on Dec. 29, 2021.

(51) Int. Cl. C12M 1/12; C12N 5/00; C12M 3/00

(52) U.S. Cl. CPC C12M 25/14; C12M 21/08; C12N 5/0068; C12N 2533/30

(57) ABSTRACT

This disclosure relates to methods of growing cells within a hydrogel scaffold and pressurizing the hydrogel and cells to induce the cells to stretch and differentiation. The disclosed method can include coating a substrate of a bioreactor with a hydrogel and seeding cells onto the hydrogel and/or the substrate. The disclosed method can further include growing the seeded cells into a cell mass and pressurizing the cell mass and the hydrogel within the bioreactor. Pressurizing the cell mass and the hydrogel induces the cell mass and hydrogel to mechanically stretch, thereby inducing hypertrophy and cell alignment.

### CROSS-REFERENCE TO RELATED APPLICATIONS

The present application claims the benefit of, and priority to, U.S. Provisional Application No. 63/294,700, entitled “METHOD FOR PRESSURIZING CELLS GROWN IN HYDROGEL INDUCE HYPERTROPHY,” filed on Dec. 29, 2021. The present application also claims the benefit of, and priority to, U.S. Provisional Application No. 63/294,703, entitled METHOD FOR WASHING AND FINISHING A GROWN CELL MASS,” filed on Dec. 29, 2021. The aforementioned applications are hereby incorporated by reference in their entirety.

### BACKGROUND

As the world’s population continues to grow, cell-based or cultured meat products for consumption have emerged as an attractive alternative (or supplement) to conventional meat from animals. For instance, cell-based, cultivated, or cultured meat represents a technology that could address the specific dietary needs of humans. Because the cells for cell-based meat are lab grown, lab methods of preparing cell-based meat can modify the profile of essential amino acids and fats and enrich such meat in vitamins, minerals, and bioactive compounds. In some cases, cell-based-meat products can be prepared from a combination of cultivated adherent and suspension cells derived from a non-human animal that facilitate such modifications and enrichment.

In addition to addressing dietary needs, cell-based-meat products help alleviate several drawbacks linked to conventional meat products for humans, livestock, and the environment. For instance, conventional meat production involves controversial practices associated with animal husbandry and slaughter. Other drawbacks associated with conventional meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based-meat products, existing methods for cultivating and processing cell-based-meat products face several shortcomings, such as challenges or failures to mimic the textures and nutritional composition of slaughtered meat. In particular, existing methods often produce meat cells with limited growth and lack of cell alignment, differentiation, and hypertrophy. In contrast to conventional meat from animals that have larger bulkier meat fibers, existing methods to grow cell-based-meats often result in smaller and weaker meat fibers. Thus, existing methods fall short of creating cell-based-meat products with textures comparable to conventional meat.

Some existing methods attempt to stretch cultivated meat cells to mimic the mechanical strain undergone by conventional meat. In some examples, existing methods use moving mechanical elements to physically stretch cultivated meat cells. However, the introduction of the moving mechanical elements required to stretch the meat cells introduce risks to sterility. Furthermore, these existing methods are often unscalable. More specifically, increasing the amount of tissue stretched by the moving mechanical elements increases the risk of contamination and the inability to control stretching pressure.

These, along with additional problems and issues are present in existing methods for cultivating cell-based meat products.

### BRIEF SUMMARY

This disclosure generally describes methods of growing cells within a hydrogel scaffold and pressurizing the hydrogel and cells to induce the cells to stretch and to induce differentiation. In particular, by applying pressure to the hydrogel and the integrated cells, the disclosed method can cause cells to form aligned musculoskeletal or cytoskeletal fibers and promote myogenic differentiation and hypertrophy. For example, the disclosed method can include preparing a hydrogel into a liquid form and spreading the hydrogel across substrates in a sealed environment, such as a bioreactor. The disclosed method can include seeding cells evenly across (or mixed throughout) the hydrogel and growing the cell-hydrogel combination for a growth period. The disclosed method further includes pressurizing the sealed environment to cause the cells (or the cell-hydrogel combination) to stretch. This cellular stretch can induce cell hypertrophy, align muscle fibers, and facilitate cell trans-differentiation into muscle tissue.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various embodiments will be described and explained with additional specificity and detail through the use of the accompanying drawings, which are summarized below.

FIGS. 1A-1B illustrate an overview diagram of pressurizing a cell mass and hydrogel to induce cell hypertrophy and alignment in accordance with one or more embodiments of the present disclosure.

FIG. 2 illustrates example techniques for hydrogel formation in accordance with one or more embodiments of the present disclosure.

FIG. 3 illustrates various techniques for hydrogel distribution in accordance with one or more embodiments of the present disclosure.

FIG. 4 illustrates utilizing various techniques for seeding cells across hydrogel in accordance with one or more embodiments of the present disclosure.

FIG. 5 illustrates pressurizing a sealed environment to stretch hydrogel and cell masses in accordance with one or more embodiments of the present disclosure.

FIGS. 6A-6C illustrate histological cross-sections indicating various benefits of stretching hydrogel and cell masses in accordance with one or more embodiments of the present disclosure.

FIG. 7 illustrates a series of acts for stretching hydrogel and cells to induce hypertrophy in accordance with one or more embodiments of the present disclosure.

### DETAILED DESCRIPTION

This disclosure describes one or more embodiments of a method for growing cells in a hydrogel scaffold to both promote cell growth and enable stretching of the cells by applying pressure to the hydrogel-cell combination. The disclosed method includes spreading and forming a hydrogel across a substrate within a growth environment. Cells can be seeded evenly across the hydrogel or the substrate and grown for a period of time. The disclosed method can further comprise pressurizing the volume surrounding the cells, whereby the hydrogel scaffold is stretched to promote hypertrophy in the cells and to enhance alignment of tissue fibers.

To illustrate, the disclosed method comprises coating a substrate with a hydrogel and seeding cells onto at least one of the hydrogel or the substrate. Such a coating can be applied as a layer on a portion of the substrate, and the seeding of cells can be spread over and through the hydrogel or mixed into a blend with the hydrogel. After seeding the cells, the disclosed method further includes growing the seeded cells into a cell mass and pressurizing the cell mass and the hydrogel within a sealed environment to induce the seeded cells to stretch.

As mentioned, the disclosed method includes forming a hydrogel. The hydrogel provides an elastic modulus favorable to cell growth. Generally, a liquid hydrogel solution can be applied to a substrate and subsequently gelated. In some embodiments, the disclosed method includes adhering the hydrogel to a substrate within a sealed environment. The hydrogel can be evenly spread across the substrate at various thicknesses to facilitate 3-dimensional cell growth while also allowing the transfer of metabolites and nutrients to the growing cells.

The disclosed method can also include seeding cells onto at least one of the hydrogel or the substrate. In some embodiments, cells are circulated over the formed hydrogel or flooded into a vessel containing the formed hydrogel. Additionally, or alternatively, the hydrogel can be processed to form a semi-liquid and mixed with cells before adherence to the substrate. Thus, the cells are attached to the hydrogel and continue growing in the hydrogel.

After layering a substrate with a hydrogel and seeding cells to grow into a cell mass, the disclosed method further includes pressurizing the cell mass. Generally, the disclosed method can comprise increasing pressure within a sealed environment to induce cells to stretch. In particular, the disclosed method can include increasing and decreasing pressure on a schedule to cause the hydrogel-cell matrix to stretch under the pressure. In some embodiments, pressurizing the cell mass comprises emitting pulses of pressure toward the cell mass within the sealed environment. In some cases, the pressure induces the cells to mechanically stretch, which facilitates cell alignment and robust muscle fiber alignment, leading to hypertrophy.

The disclosed method provides several benefits relative to unprocessed adherent cell cultures or other existing and unprocessed cell-based meats. In particular, by stretching the hydrogel-cell matrix, the disclosed method influences cytoskeletal signaling by mechanotransduction to enhance cell growth, differentiation, and activity. Generally, stretching the hydrogel-cell matrix mimics forces undergone by conventional meat during exercise. Thus, cells in the resulting cell-based product share more properties with conventional meat cells relative to unprocessed meat cells not exposed to a stretching mechanism. More specifically, the pressurization of the cell mass can cause cells to form aligned musculoskeletal or cytoskeletal fibers. Furthermore, stretching the hydrogel-cell matrix can promote myogenic differentiation and hypertrophy, which may promote the formation of larger and bulkier meat fibers.

The disclosed method further eliminates sterility risks that hinder existing methods. In contrast to existing methods that rely on moving mechanical elements to stretch cells, the disclosed method combines the use of a hydrogel together with pressure in a sterile environment to eliminate the sterility risk associated with the utilization of mechanical parts for stretching meat. Furthermore, the disclosed method can comprise various techniques for coating substrates within a sealed growth environment with hydrogel. Thus, in some embodiments, the disclosed method includes sterilizing the hydrogel once and growing cells within the same environment to further reduce the risk of contamination

In addition to improved sterility, the disclosed method is scalable to efficiently stretch greater volumes of cells relative to existing methods. As mentioned, the disclosed method applies mechanical stimulation by pressurizing a sealed environment, such as a bioreactor. The disclosed method can include pressurizing a vast array of tissues in a single or multiple connected chambers. Thus, the hydrogel-cell matrix can be stretched with shared equipment at a significantly reduced expense.

As illustrated by the foregoing discussion, the present disclosure utilizes a variety of terms to describe features and advantages of the disclosed method. Additional detail is now provided regarding the meaning of such terms. For example, as used herein, the term “cell mass” refers to a mass comprising cells of meat. In particular, a cell mass refers to cells of cultured meat gathered into a collective mass. As discussed below, a cell mass may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, or other similar cell types. For example, a cell mass can include a cell sheet of cultured meat growing within an enclosure, such as a chamber, housing, container, etc.

Relatedly, the term “growing cell mass” refers to a cell mass comprised of one or more growing cells. For instance, a growing cell mass includes a group of cells nourished by a growth medium to grow during a growing time period.

As further used herein, the term “cells” refer to individual cells of meat. In particular, cells may comprise different cell types, such as one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, and other similar cell types. Furthermore, cells may comprise different types of progenitor cells including myogenic progenitors, adipogenic progenitors, mesenchymal progenitors, or other types of progenitor cells.

As also used herein, the term “substrate” refers to a material on which cells grow. In particular, a substrate includes a material to which cells or a hydrogel adhere and upon which cells grow. Accordingly, a substrate can support or promote cell adhesion, cell differentiation, and/or growth of cells to form a cell mass-namely, a comestible meat product. For example, a steel substrate or other substrate can be positioned to receive cultured cell media as part of a seeding process inside a bioreactor. Once the cell mass grows to a predetermined size or for a predetermined duration, the cell mass is harvested from the substrate. The substrate can include a variety of bio-compatible materials, such as a metal material or polymer material.

As used herein, the term “hydrogel” refers to a crosslinked hydrophilic polymer that does not dissolve in water. In particular, a hydrogel can adhere to a substrate to support or promote the growth of cells. For example, a hydrogel can be animal based (e.g., bovine gelatin, alginate, etc.) or synthetic (e.g., recombinant collagen, fibronectin, etc.).

As used herein, the term “hydrogel” refers to a crosslinked hydrophilic polymer that retains integrity and mass in water based cell culture conditions, and may be induced to solubilize or otherwise change structure by change in temperature, pH, ionic concentration, enzymatic activity or chemical reaction. In particular, a hydrogel can adhere to a substrate to support or promote the growth of cells. For example, a hydrogel can be animal based (e.g., bovine gelatin, alginate, etc.) or synthetic (e.g., recombinant collagen, fibronectin, etc.).

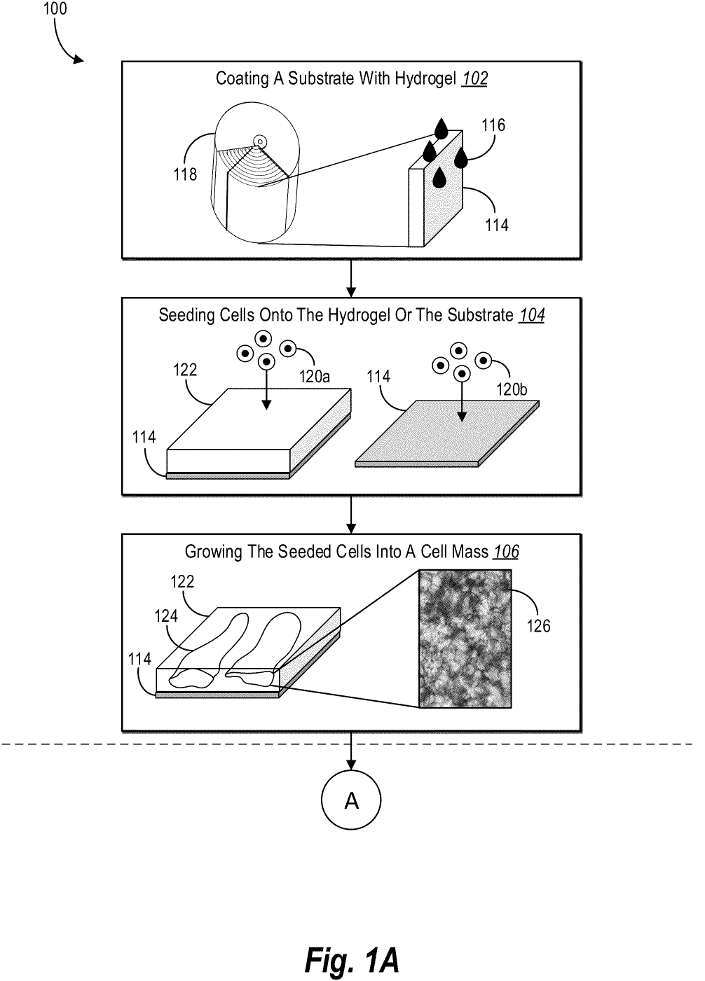

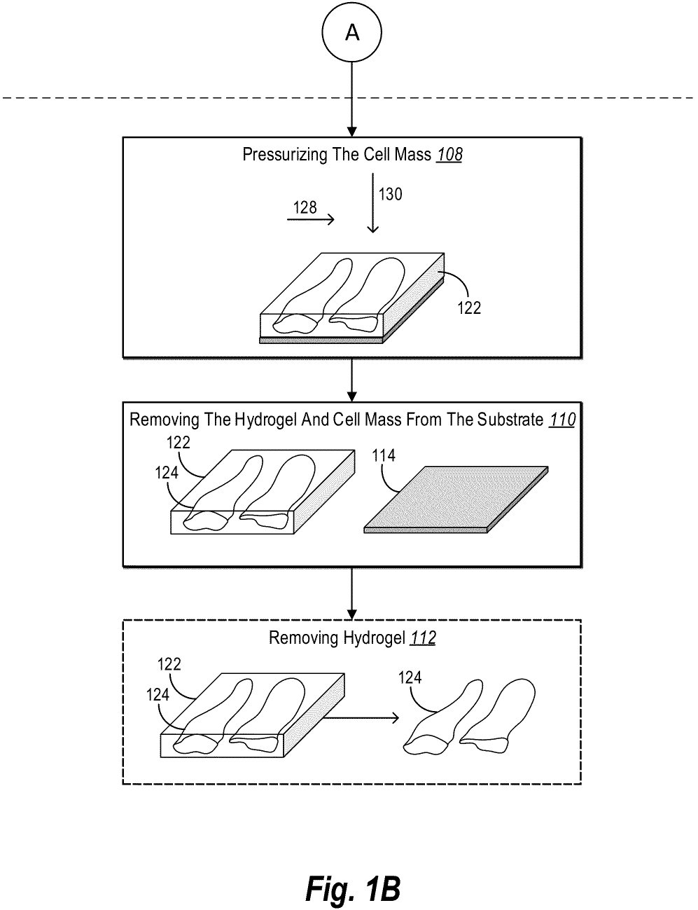

Additional detail will now be provided regarding a disclosed method in relation to illustrative figures portraying example embodiments and implementations of the disclosed method. For example, FIGS. 1A-1B illustrate a series of acts 100 for pressurizing a cell mass to induce cells to stretch. In particular, in some embodiments, the series of acts 100 includes an act 102 of coating a substrate with hydrogel, an act 104 of seeding cells onto the hydrogel or the substrate, an act 106 of growing the seeded cells into a cell mass, an act 108 of pressurizing the cell mass, an act 110 of removing the hydrogel and cell mass from the substrate, and an optional act 112 of removing hydrogel.

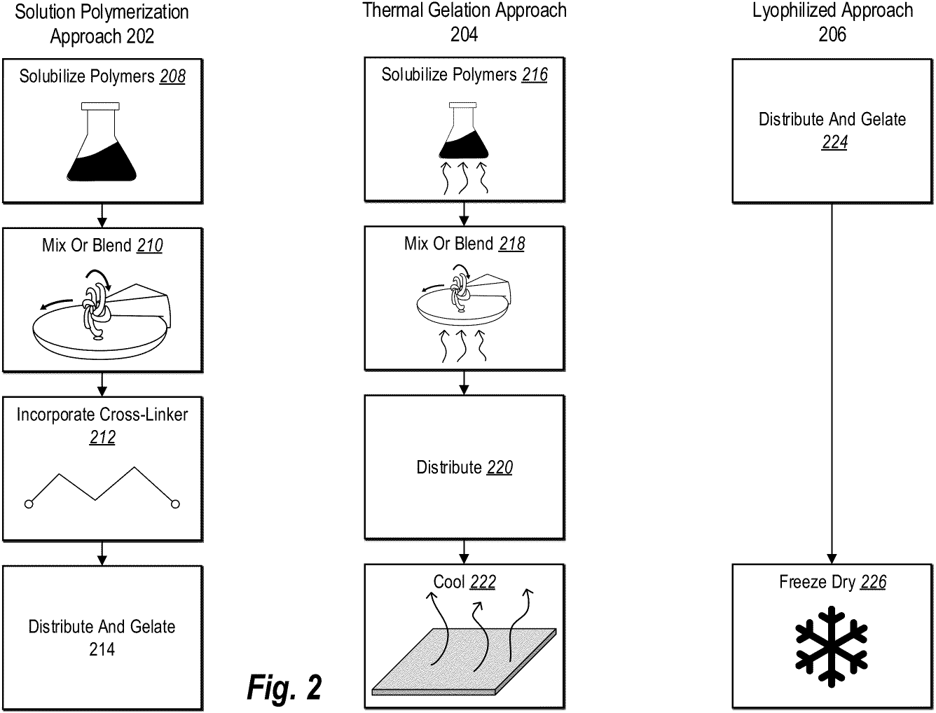

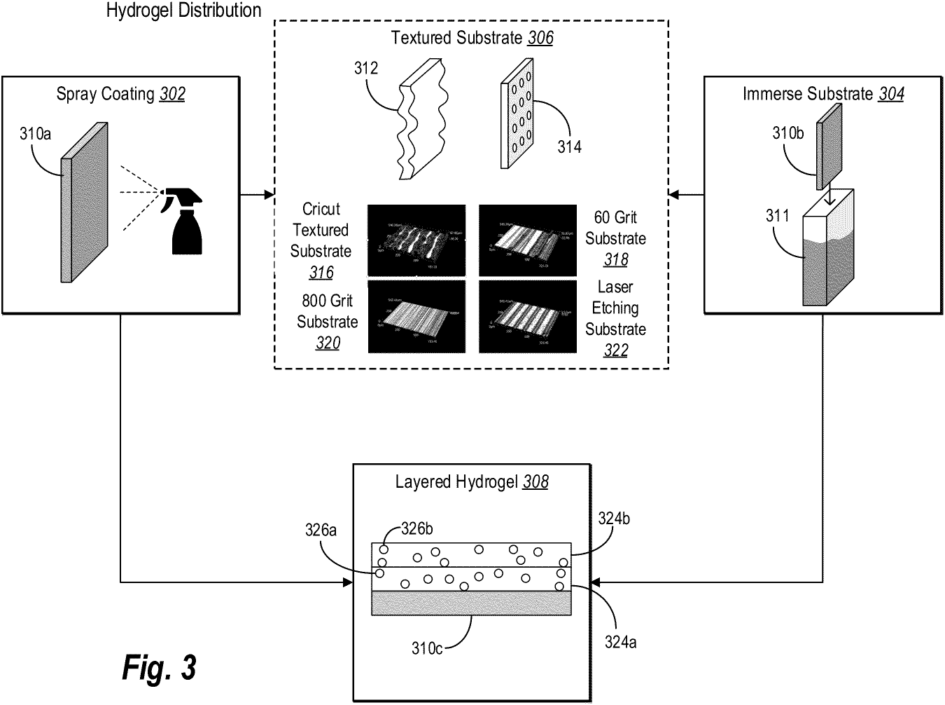

As illustrated in FIG. 1A, the disclosed method includes the act 102 of coating a substrate with hydrogel. In particular, the disclosed method includes applying a hydrogel solution 116 to a substrate 114 within a sealed environment 118. In some examples, the hydrogel covers the entire substrate. Alternatively, in some embodiments, the hydrogel covers part or a portion of the substrate 114. As indicated above, in some cases, the sealed environment 118 is a bioreactor. FIGS. 2-3 and the corresponding paragraphs detail various techniques by which the hydrogel solution 116 is spread across the substrate 114 and gelated in accordance with one or more embodiments.

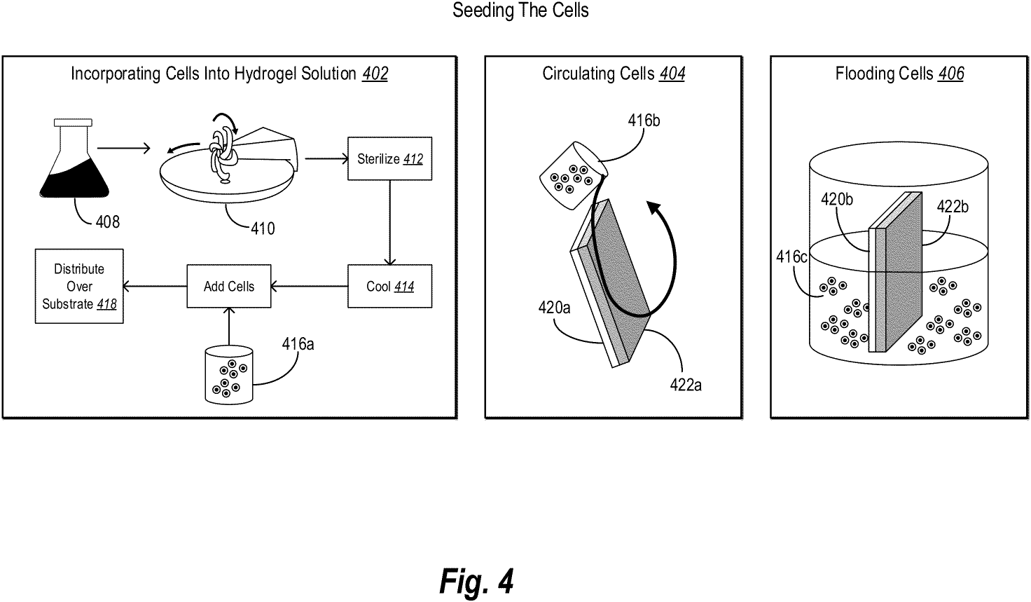

FIG. 1A further illustrates the act 104 of seeding cells onto the hydrogel or the substrate. Generally, the disclosed method can include seeding cells 120a onto a hydrogel 122. Additionally, or alternatively, the disclosed method includes seeding cells 120b directly onto the substrate 114. For example, the disclosed method can comprise mixing the cells 120b with a hydrogel solution (e.g., the hydrogel solution 116) and coating the substrate 114 with the blend of hydrogel and cells. FIG. 4 and the corresponding paragraphs detail various techniques for seeding the cells onto the hydrogel or the substrate in accordance with one or more embodiments.

As an alternative to coating the substrate with the hydrogel, the disclosed method can utilize a hydrogel to grow a cell mass in a suspension system. In particular, the suspension system can suspend the hydrogel using the hydrogel as a carrier. The suspension system can further form hydrogel particles that can be suspended as a fluid gel. To create the fluid gel, the solidified or gelated hydrogel is mechanically disrupted by blending or mixing to produce a dispersion of particles with irregular surfaces. When such particles are dispersed, the dispersed mixture has positive organoleptic properties and is associated with a velvety texture.

The hydrogel may be blended before or after sterilization. The hydrogel particles can be introduced into an agitated bioreactor (e.g., stirred, rocked, airlifted, recirculated, etc.) along with cells and culture media. The cells would, over time, collide with the hydrogel particles, attach, then spread and grow across the surface of or into the hydrogel particles.

As illustrated in FIG. 1A, the series of acts 100 includes the act 106 of growing the seeded cells into a cell mass. Generally, the disclosed method includes incubating the cells for a growth period to form a cell mass. As illustrated, the cells have grown into a cell mass 124 within the hydrogel 122 adherent to the substrate 114. As further illustrated by a hydrogel cross section 126 in FIG. 1A, the hydrogel scaffold supports growth of cells. More specifically, the hydrogel cross section 126 is stained to show cell adhesion through the hydrogel scaffold.

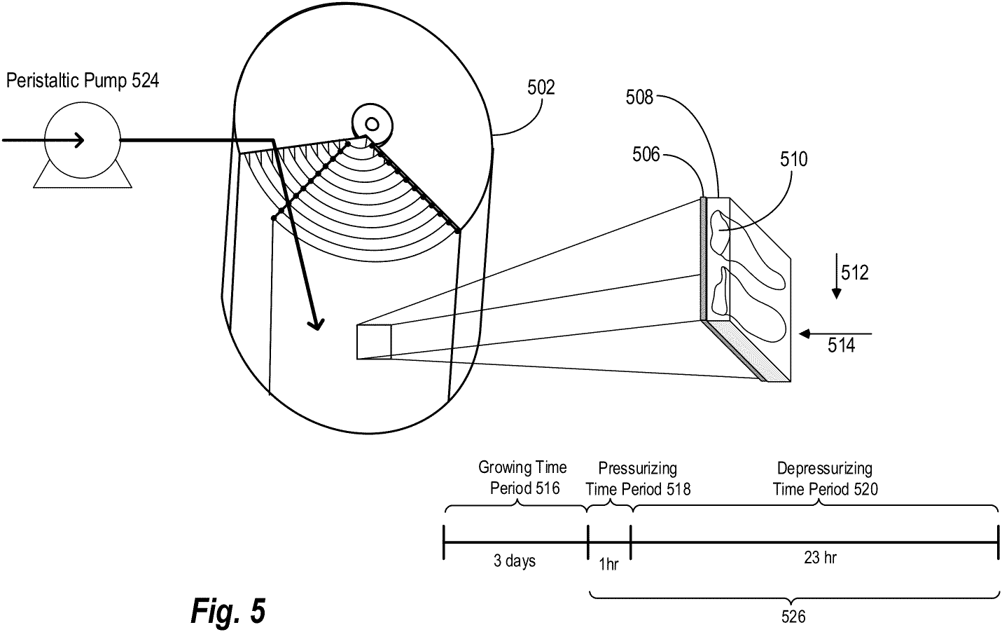

FIG. 1B illustrates the act 108 of pressurizing the cell mass. In particular, and as illustrated, the disclosed method includes pressurizing the sealed environment to induce cells to stretch when within or on a hydrogel matrix. In some embodiments, the disclosed method includes creating a pressure gradient across a height 130 of the hydrogel 122 and a pressure gradient across a length 128 of the hydrogel 122. FIG. 5 and the corresponding discussion provide additional detail regarding how the sealed environment can be pressurized to induce the cells (or the cell-hydrogel matrix) to stretch.

FIG. 1B further illustrates the act 110 of removing the hydrogel and the cell mass from the substrate. Generally, the disclosed method includes removing the hydrogel 122 and the cell mass 124 from the surface of the substrate 114. The disclosed method can include various hydrogel removal methods. For example, the cell mass 124 and the hydrogel 122 can be mechanically separated from the substrate 114 by a fluid flow at a rate (e.g., 10 feet per second) sufficient to mechanically dislodge the cell mass 124 and the hydrogel 122 from the substrate 114. In some embodiments, the sealed environment and/or the substrate 114 can be heated to release the hydrogel 122 via melting and/or partial melting. The disclosed method may include other techniques for removing the hydrogel and the cell mass from the substrate. In some embodiments, the complexed cells and the hydrogel can be prepared together as a comestible meat product.

As further illustrated in FIG. 1B, the series of acts 100 may include the optional act 112 of removing the hydrogel. In particular, the cell mass 124 and the hydrogel 122 can be further processed to isolate the cell mass 124. The disclosed method can include various hydrogel removal techniques. Generally, the optional act 112 includes steps to reverse the formation or gelation of the hydrogel to solubilize the hydrogel. Various hydrogel formation techniques are described with respect to FIG. 2.

When performing the act 112, for example, the hydrogel 122 can be removed using a thermal method, electrostatic method, enzymatic method, pH method, or a degradable resorbable scaffold method. For example, if the hydrogel 122 is formed/gelated using thermal techniques (i.e., cooling), then the hydrogel 122 can be heated to solubilize the hydrogel 122 back to solution. The cell mass 124 may subsequently be separated from the hydrogel solution by centrifugation, filtration, or settling.

In yet another example of the optional act 112, the disclosed method can include an electrostatic technique for reversing gelation. For example, polyanionic polymer networks within the hydrogel 122 when composed of alginate may be stabilized by the addition of divalent cations such as calcium (Ca2+) to form a hydrogel. To solubilize the polymer and release the cell mass 124, the disclosed method includes removing the cations with the addition of a chelator, such as Ethylenediaminetetraacetic acid (EDTA). The cell mass 124 can be further separated from the hydrogel solution by centrifugation, filtration, or settling.

As further mentioned, the disclosed method may include an enzymatic method for solubilizing hydrogels as part of performing the optional act 112. In particular, a protein hydrogel may be decomposed using an enzyme (e.g., collagenase). The disclosed method may also include a pH method for solubilizing the hydrogel 122. In particular, the disclosed method might include changing pH to dissolve the hydrogel 122. For example, collagen-based hydrogels may be dissolved by reducing the pH in solution to release the cell mass 124.

Additionally, or alternatively, the disclosed method can include using a degradable or resorbable hydrogel. For example, if the hydrogel 122 is a composition that is degradable by the cells or conditions within the sealed environment 118, the hydrogel 122 could be replaced by the cell mass 124 during the growth period. For example, the cells within the cell mass 124 could metabolize or otherwise degrade the hydrogel 122. Any remaining hydrogel mass may then be removed with the growth media.

As mentioned, the disclosed system may include various techniques for forming a hydrogel. Generally, the disclosed method can implement various hydrogel compositions that require different conditions for gelation. FIG. 2 and the corresponding discussion detail various hydrogel formation techniques in accordance with one or more embodiments. In particular, FIG. 2 illustrates a solution polymerization approach 202, a thermal gelation approach 204, and a lyophilized approach 206.

Regardless of the approach for forming a hydrogel, the disclosed method can include the formation of hydrogels of various compositions. Generally, the hydrogel can be natural or synthetic. Examples of naturally derived hydrogels may include bovine gelatin, alginate, chitosan, cellulose, chemically modified versions of cellulose, or other natural polymers. Examples of synthetic hydrogels include recombinant agriculturally relevant gelatin, recombinant collagen, fibronectin, laminin, other extracellular matrix proteins including glycosaminoglycans and hyaluronic acid, or other synthetic polymers. In some embodiments, the structural polymer of the hydrogel is supplemented with additional functional molecules, such as adhesion factors, growth factors, nutrients (e.g., minerals and vitamins), or other additives.

As illustrated in FIG. 2, the disclosed method utilizes the solution polymerization approach 202. In some embodiments, and as illustrated, the solution polymerization approach 202 includes a step 208 of solubilizing polymers, a step 210 of mixing or blending, a step 212 of incorporating cross-linkers, and a step 214 of distributing and gelating. In particular, the step 208 comprises solubilizing hydrogel polymers in a suitable solvent. Example solvents can include water, ethanol, water-ethanol mixtures, benzyl alcohol, or other solvents. The solubilized polymers can be mixed or blended to ensure a homogenous solution.

The solution polymerization approach 202 also includes the step 212 of incorporating cross-linker. Cross-linkers can include transglutaminase, genipin, or other enzymes or compounds. The solution polymerization approach 202 can include controlling the concentration of cross-linkers to control the working time of the hydrogel solution prior to gelation. The solution polymerization approach 202 further includes the step 214 of distributing and gelating, which comprises distributing the hydrogel solution over a target substrate and allowing the hydrogel solution to gelate.

In addition or in the alternative to the solution polymerization approach 202, FIG. 2 further illustrates the thermal gelation approach 204. In particular, the thermal gelation approach 204 includes a step 216 of solubilizing polymers, a step 218 of mixing or blending, a step 220 of distributing, and a step 222 of cooling. In further alternatives, the thermal gelation approach begins with cooling the gel and finishes the gel by heating. The step 216 includes solubilizing hydrogel polymers in a suitable solvent (e.g., water, ethanol, benzyl alcohol, etc.). In some embodiments, and as illustrated, the polymers are solubilized at an elevated temperature (e.g., 40-50 C). The step 218 of mixing or blending and the step 220 of distributing the hydrogel solution over a substrate also occur at an elevated temperature (e.g., 40-50 C). The thermal gelation approach 204 further includes the step 222 of cooling the hydrogel solution. In particular, the disclosed method includes reducing the temperature of the hydrogel solution, the substrate, and/or the sealed environment to induce thermal gelation. In the alternative (or in addition) to colling the hydrogel solution, in some embodiments, the disclosed method heats or increases the temperature of the hydrogel solution.

As further illustrated in FIG. 2, the disclosed method can include the lyophilized approach 206. Generally, the lyophilized approach 206 is utilized to produce a porous structure in a gelated hydrogel. In particular, the lyophilized approach 206 comprises a step 224 of distributing and gelating a hydrogel solution and a step 226 of freeze drying the hydrogel. The lyophilized approach 206 can be used in conjunction with some or all of the solution polymerization approach 202 and/or the thermal gelation approach 204. For example, the step 224 comprises distributing and gelating a hydrogel formed using some or all of the solution polymerization approach 202, optionally including cross-linker(s), and/or the thermal gelation approach 204. In some embodiments, the lyophilized approach 206 includes allowing the hydrogel solution to gelate for a time period (e.g., 10-120 minutes, 1-24 hours, etc.). As further illustrated, the lyophilized approach 206 includes the step 226 of freeze drying the hydrogel.

The step 226 produces a porous structure in the gelated hydrogel. For example, the hydrogel can be freeze dried or vacuum dried to pull out water from the hydrogel to form channels and pores in the hydrogel. Channels and pores in the hydrogel are particularly beneficial because they facilitate cell growth, enable cells to penetrate deeply into the layer, facilitate 3-dimensional cell growth, accelerate cell differentiation, and form a thicker layer of tissue than would be possible on a 2-dimensional sheet. Furthermore, by increasing the porosity of the hydrogel, the lyophilized approach 206 increases the surface area of the hydrogel which improves the ratio of cell to hydrogel ratio of the final cell-based product.

As indicated above, the disclosed method can include a combination of the solution polymerization approach 202, the thermal gelation approach 204, and the lyophilized approach 206. The solution polymerization approach 202 and thermal gelation approach 204 support the incorporation of live cells within the hydrogel, and the incorporation of thermally sensitive bioactive molecules that may not survive lyophilization. For example, and as mentioned, the lyophilized approach 206 can be used in combination with the solution polymerization approach 202 and/or the thermal gelation approach 204. Furthermore, in some embodiments, the solution polymerization approach 202 can be used in conjunction with the thermal gelation approach 204. To illustrate, the disclosed method can include the use of both cross-linkers and temperature variation to form hydrogels.

In some embodiments, the disclosed method comprises distributing and gelating the hydrogel within a sealed environment. In particular, the disclosed method can include forming a hydrogel utilizing the solution polymerization approach 202, the thermal gelation approach 204, and the lyophilized approach 206 within a bioreactor. FIG. 3 and the corresponding discussion provide additional detail regarding how a hydrogel can be added to a substrate. In some embodiments, the hydrogel solution is added to a substrate within a bioreactor and gelated within the bioreactor.

In one or more embodiments, the disclosed method includes the formation of a heat-stable hydrogel. In particular, the solubilized polymers described with respect to one or more of the solution polymerization approach 202, the thermal gelation approach 204, and the lyophilized approach 206 include the use of a heat-stable hydrogel polymer. In particular, heat-stable hydrogel polymers are stable through temperatures required for sterilization (e.g., 121 F) without a loss of functional properties. For example, in some embodiments, the hydrogel solution is added to a bioreactor and sterilized using heat. The hydrogel solution can then be spread across substrates within the bioreactor and gelated using temperature and/or the addition of cross-linkers. More specifically, the cross-linkers may be filtered, sterilized, then injected into the bioreactor to gelate the hydrogel solution. Thus, the disclosed method includes techniques for maintaining a sterile environment within the bioreactor.

After forming or collecting a hydrogel, in some embodiments, the disclosed method distributes the hydrogel on a substrate. FIG. 3 and the corresponding paragraphs detail techniques for coating a substrate with a hydrogel. In some embodiments, the disclosed method comprises evenly spreading the hydrogel across a substrate to support cell growth. The disclosed method may accomplish this by spray coating a substrate or immersing the substrate in hydrogel solution. In some embodiments, the disclosed method includes coating a substrate by spray coating 302 the substrate. For example, and as illustrated in FIG. 3, the disclosed method can include spray coating 302 a substrate 310a with a hydrogel solution. In particular, the hydrogel solution may be ejected from one or more nozzles at the substrate 310a configured for cell adhesion and growth. The nozzles may rotate, oscillate, or otherwise move to ensure even distribution across the substrate 310a.

As further illustrated in FIG. 3, the disclosed method may comprise immersing a substrate 304. In particular, and as illustrated, immersing a substrate 304 may comprise dipping a substrate 310b in a hydrogel solution 311. The substrate 310b may be dipped into the hydrogel solution 311 for the duration of a coating period and removed from the hydrogel solution 311. In some embodiments, instead of dipping the substrate 310b, which requires moving the substrate 310b, the disclosed method comprises flooding a vessel (e.g., a bioreactor) containing the substrate 310b with a hydrogel solution. The hydrogel solution can be drained at a controlled rate to deposit hydrogel solution onto the surface of the substrate 310b. Accordingly, a vessel (e.g., a bioreactor) can be drained of the hydrogel solution.

When utilizing spray coating 302 or immersing a substrate 304, the disclosed method can control the thickness of the distributed hydrogel. The thickness of the distributed hydrogel can be controlled through the width/gap size of substrate 304 in a vessel such as the bioreactor 502. Additionally, desired thicknesses could be achieved by layering hydrogel solution onto the substrate once the initial layers of hydrogel solution have gelated. In some embodiments, the disclosed method comprises controlling the thickness of the hydrogel based on hydrogel properties. For example, the hydrogel solution may include thickening agents. Generally, if a hydrogel is too thick, the mass transfer of metabolites and nutrients is limited and cells located further from the hydrogel-growth media interface will face suboptimal growth conditions. In contrast, hydrogels that are too thin are quickly outgrown by cells. Furthermore, thin hydrogels put cells in direct contact with the substrate and fail to mechanically shield the cells during cell-hydrogel stretch. In one or more embodiments, the disclosed method forms a hydrogel that is 1 mm thick. In other embodiments, the disclosed system creates a hydrogel with a thickness between 0.005 mm and 10 mm thick, and preferably may be between 1 and 2 mm thick.

In some embodiments, the disclosed method improves adhesion of the hydrogel to the substrate by utilizing a textured substrate 306. In particular, the textured substrate 306 can increase gel adherence by causing the hydrogel to bind tightly enough to avoid slipping off the substrate. The substrate may be textured in such a way to enhance cell adhesion while still enabling the hydrogel to be removed from the substrate after the growth stage. For example, the disclosed method can include the utilization of a wavy substrate 312 or a porous substrate 314. Generally, the wavy substrate 312 has channels that form ridges or columns of hydrogel. The ridges or columns of hydrogel may provide additional structure for the grown cell mass and consequently improve the texture of the final cell-based meat product. For example, columns of hydrogel (and grown cell mass) may provide additional surfaces that increase the amount of bite resistance when cooked and consumed. FIG. 3 further illustrates some examples of designs for the wavy substrate 312. Generally, the spacing, size (e.g., width, length, depth), and frequency of waves or channels within the wavy substrate 312 may be customized. For example, FIG. 3 illustrates a cricut textured substrate 316, a 60 grit substrate 318, an 800 grit substrate 320 and a laser etching substrate 322.

Furthermore, and as illustrated in FIG. 3, the disclosed method may comprise utilization of the porous substrate 314. The hydrogel, when spread across the porous substrate 314 will form columnar structures. For example, the porous substrate 314 may contain parallel channels 50-1000 micrometers wide and 10-1000 micrometers deep and separated by distances of 10-1000 micrometers. As the hydrogel gelates against the porous substrate 314, the hydrogel makes channels that form noodle-like structures that may be similar to meat fibers found in conventional meat. For example, similar to how meat fibers have several surfaces that provide resistance for teeth to break through during chewing, the noodle-like structures provide numerous surfaces that must be broken during chewing thereby providing increased resistance within a cell-based meat product and a more complex texture. In one or more embodiments, the disclosed method uses substrates with different textures. For example, a textured substrate may include ridges, grooves, channels, and pores to improve cell adhesion. Additional patterned textures are described in U.S. Pat. Pub. No. 2021/0106032 A1, entitled APPARATUSES AND METHODS FOR PREPARING A COMESTIBLE MEAT PRODUCT, filed on Dec. 22, 2020, the contents of which are expressly incorporated herein by reference.

In addition to different textures, the substrate can include a variety of different materials. To illustrate, in one or more embodiments, the substrate comprises one or more of polylactic acid, starch derived materials, waxes (e.g., paraffin, beeswax), oils (e.g., food derived substance like coconut oil), polychlorotrifluoroethylene, polyetherimide, polysulfone, polystyrene, polycarbonate, polypropylene, silicone, polyetheretherketone, polymethylmethacrylate, nylon, acrylic, polyvinylchloride, vinyl, phenolic resin, petroleum-derived polymers, glass, polyethylene, terephthalate, titanium, aluminum, cobalt-chromium, chrome, silicates, glass, alloys, ceramics, carbohydrate polymer, mineraloid matter, and combinations or composites thereof.

In particular embodiments, the substrate includes stainless steel (e.g., an austenitic stainless steel, a ferritic stainless steel, a duplex stainless steel, a martensitic and precipitation hardening stainless steel, a passivated stainless steel). For example, the substrate includes food grade stainless steel, such as Grade 316 stainless steel, or Grade 430 stainless steel (e.g., for enhanced corrosion resistance). Alternatively, the substrate includes a super elastic or shape-memory material (e.g., a nickel titanium alloy, Nitinol) that retains or can revert back to a predetermined shape. In certain implementations, metal materials can provide increased cleanability and/or sterilization. In contrast, polymer materials can provide increased cell adhesion properties and facilitate additional manufacturing methods (e.g., injection molding or extrusion) not available to certain metals.

As further illustrated in FIG. 3, the disclosed method can include creating a layered hydrogel 308. In particular, in certain implementations, the disclosed method comprises coating the substrate 310c in layers of hydrogel 324a-324b. To form the layered hydrogel 308, the substrate can be dipped or spray coated with a first hydrogel solution, the first hydrogel solution is gelated to form the first layer of hydrogel 324a, and the first layer of hydrogel 324a can be seeded with cells 326a. The hydrogel 324a can cover the whole or a portion of the substrate 310c. The substrate 310c and the first layer of hydrogel 324a can then be further coated by the second layer of hydrogel 324b by either spray coating or immersing the substrate and the first layer of hydrogel 324a in a second hydrogel solution. The second hydrogel solution is gelated and seeded with cells 326b. The hydrogel 324b can cover the whole or a portion of the hydrogel 324a.

In some embodiments, the combined thickness of the layers of hydrogel 324a-324b equals a determined gel thickness. For example, the combined thickness of the layers of hydrogel 324a-324b can equal 0.005 mm to 10 mm. In some embodiments, the first layer of hydrogel 324a and the second layer of hydrogel 324b are equal in thickness (e.g., both are 0.5 mm). In other embodiments, the first layer of hydrogel 324a is thicker or thinner than the second layer of hydrogel 324b. For example, the first layer of hydrogel 324a can equal 0.6 mm while the second layer of hydrogel 324b is 0.4 mm.

As indicated above, in some embodiments, the layers of hydrogel 324a-324b are seeded with different cell types. For example, the cells 326a can comprise a different cell type than the cells 326b. Alternatively, the cells 326a can comprise a different mixture of cell types than the cells 326b. In some cases, the cells 326a-326b can comprise one or more of myoblasts, mesangioblasts, myofibroblasts, mesenchymal stem cells, hepatocytes, fibroblasts, pericytes, adipocytes, epithelial, chondrocytes, osteoblasts, osteoclasts, pluripotent cells, somatic stem cells, endothelial cells, and other similar cell types. The different hydrogel layers can be seeded with different cell types to create an organized tissue where the defined layers are designed to influence tissue quality or increase the productivity of the disclosed method. Furthermore, the proportion and the relative position of cell types with each other can be defined by the layering process.

As illustrated in FIG. 3, the layered hydrogel 308 comprises two layers of hydrogel attached to the substrate 310c. In some implementations, the disclosed method includes coating the substrate 310c with three or more layers of hydrogel. Each of the layers of hydrogel can be seeded with different cell types. In one example, three separate hydrogel layers each include one of fibroblast, myoblast, and adipocyte cells where the cells are provided in ratios that, once fully grown, mimic a target conventional meat. In addition to providing support for the growth of multiple cell types, the inclusion of several layers of hydrogel may improve the texture of the final cell-based product by giving additional surfaces to bite through to provide a familiar chewing experience. In particular, by including different layers of hydrogel, the disclosed method forms different structural filaments that mimics the filamentous structure typical of most conventional meats.

As suggested above, in some embodiments, the disclosed method does not include creating a layered hydrogel. Instead, the disclosed method can include spreading a single hydrogel layer across a substrate and seeding cells in the single hydrogel layer. In some such embodiments, the disclosed method can seed different types of cells on the single hydrogel layer, including, but not limited to, the cell types just listed above.

As mentioned previously, in certain implementations, a hydrogel scaffold supports the growth of a thick cell mass or tissue sheet. FIG. 4 and the corresponding paragraphs describe different techniques for seeding cells onto the hydrogel or the substrate in accordance with one or more embodiments. In particular, FIG. 4 illustrates incorporating cells into a hydrogel solution 402, circulating cells 404 over a hydrogel, and flooding cells 406 over a hydrogel.

As illustrated in FIG. 4, one way by which cells are seeded into a hydrogel is by incorporating the cells into a hydrogel solution 402. Generally, a hydrogel solution 408 may be blended 410, sterilized 412, cooled 414, and cells 416a added. The blend of hydrogel and cells can then be distributed over a substrate 418. More specifically, in certain implementations, the hydrogel solution 402 is blended for a time (e.g., 60 seconds, 10 minutes, etc.) until it forms a homogeneous solution. A properly blended hydrogel solution can be characterized by a uniform appearance and consistent light scattering as measured by a turbidity meter. As further illustrated in FIG. 4, the blended hydrogel solution can be sterilized 412 by heat or filtration. The blended hydrogel solution can be cooled 414 to a temperature that can support cell growth. In an example where the hydrogel solution is sterilized by heat, the hydrogel solution is cooled to a temperature that is not harmful to cells. The cells 416a are added to the cooled hydrogel solution and distributed over the substrate.

In one or more embodiments illustrated in FIG. 4, the blend of hydrogel and cells is added over a substrate using the techniques described in relation to FIG. 3. The hydrogel within the blend of hydrogel and cells can further be gelated using techniques described with respect to FIG. 2. For example, the blend of hydrogel and cells can be thermally gelated and/or exposed to cross-linkers to form a crosslinked hydrogel-cell matrix. In further embodiments, UV light may be used to crosslink a hydrogel matrix. In some embodiments, instead of distributing the blend of hydrogel and cells over a substrate, the disclosed method includes forming the blend of hydrogel and cells into shapes.

In addition or in the alternative to incorporating the cells into a hydrogel solution, FIG. 4 further illustrates seeding cells onto a hydrogel by circulating the cells 404 over a hydrogel. In particular, the disclosed method comprises circulating the cells 416b over a hydrogel 422a attached to a substrate 420a. In particular, the cells 416b are circulated with a determined linear velocity for a circulation period for the cells 416b to impact the hydrogel 422a surface and attach. For example, the cells 416b may be circulated with a linear velocity of less than 50 cm per second over a circulation period of 24-48 hours.

Furthermore, the disclosed system can include a technique of flooding cells 406 over a hydrogel. Generally, the disclosed method can comprise submerging the hydrogel 422b (and the substrate 420b) in a cell suspension containing the cells 416c. For example, the hydrogel 422b can be submerged in the cell suspension for an attachment period and removed after the attachment period. The cells 416c may attach to the hydrogel 422b during the attachment period. In some embodiments, and as illustrated in FIG. 4, the hydrogel 422b is immersed in the cell solution for 5 minutes to 24 hours to allow the cells 416c to seed or attach. Whether immersed in a cell solution or a recipient of cell circulation or other seeding method, in some embodiments, the substrate 420b and the hydrogel 422b are fixed within a sealed environment. The sealed environment is flooded with the cell solution and flow stopped for the attachment period then removed after the attachment period.

Each of the techniques illustrated in FIG. 4 can be performed within a sealed environment, such as a bioreactor. In particular, the substrate and the hydrogel onto which the cells are seeded may be located within a sealed environment. In some embodiments, the sealed environment is sterilized prior to the addition of cells. For example, a bioreactor containing the substrates, and/or the hydrogel can be sterilized by solution (e.g, using a sodium hydroxide solution) or heat. The bioreactor can be washed and cooled prior to the seeding of cells as to avoid harming the cells.

Regardless of the seeding method depicted in FIG. 4, in one or more embodiments, the cells 416a-416c are grown in suspension before being seeded onto the hydrogel or the substrate. In particular, the cells are grown in suspension for a suspension period, until reaching a determined cell density, or until they reach a determined growth stage. In some embodiments, cells are grown in suspension for a suspension phase (e.g., 1 week, 2 weeks, etc.). Additionally, or alternatively, the cells are grown in suspension until reaching a determined cell density. For example, cells may be grown in suspension until reaching a density of 3 million cells per mL. In some embodiments, cells are grown in suspension until reaching the end of an exponential growth rate phase.

When grown in suspension, the cells 416a-416c can further be diluted or concentrated before attaching to the hydrogel or substrate. In particular, cells may be diluted in a cell suspension with growth medium. Cells may be concentrated by cell concentration methods including centrifugation, density separation, electromagnetic separation, or acoustic separation.

As previously mentioned, after seeding the hydrogel with cells, the disclosed method can comprise stretching the cells within a hydrogel matrix. FIG. 5 and the accompanying paragraphs describe how the disclosed method induces seeded cells to stretch within a hydrogel by pressurizing a sealed environment in accordance with one or more embodiments.

In particular, the disclosed method comprises pressurizing the cell mass and the hydrogel within a sealed environment for a pressurizing time period (or a first segment of time) to induce seeded cells to stretch. As illustrated in FIG. 5, the disclosed method comprises pressurizing a volume surrounding a cell mass 510 whereby the hydrogel 508 scaffold is stretched to promote hypertrophy in the cells and to enhance alignment of tissue fibers. In particular, FIG. 5 illustrates pressurizing a bioreactor 502 by utilizing a peristaltic pump 524 or a mass flow controller, although other mechanisms are contemplated.

In some embodiments, the disclosed method includes pressurizing the bioreactor 502. In particular, the disclosed method comprises pressurizing the bioreactor 502 by flow from one or more locations to limit pressure variation associated with headloss. For example, the disclosed method includes utilizing the peristaltic pump 524 to create a pressure gradient within the bioreactor 502. The peristaltic pump 524 can be located at the top of the bioreactor 502 and pump fluid into the bioreactor 502 to create flow pressure. More specifically, the peristaltic pump 524 applies pressure to process fluid (e.g., media) by increasing the flow rate against a set flow resistance, maintaining a flow rate, and changing the resistance to flow, or a combination thereof, whereby the flow provides at least intermittent pressure to intermittently stretch the hydrogel 508.

As depicted in FIG. 5, in some cases, the disclosed method comprises creating multiple pressure gradients. Generally, when the cell mass 510 is compressed along one axis, the cells expand along other faces. The disclosed method comprises creating a pressure gradient within the bioreactor 502 where the pressure at the bottom of the bioreactor 502 is greater than the pressure at the top of the bioreactor 502. Thus, the disclosed method creates a pressure gradient across a length 512 of the hydrogel 508. Pressurizing the bioreactor 502 also creates a pressure gradient across a height 514 across of the hydrogel 508 relative to a vertical axis of the hydrogel 508. In particular, surface cells within the cell mass 510 closer to the surface of the hydrogel 508 and cells closer to the pressure source (e.g., the peristaltic pump 524) experience greater pressure than cells closer to a substrate 506 and further from the pressure source.

As indicated above, in some embodiments, the substrate 506 is a metal substrate (e.g., steel) and the hydrogel 508 comprises different layers. As indicated by the bioreactor 502 and a zoomed-in depiction of the substrate 506 in FIG. 5, for example, the substrate 506 comprises a metal sheet with a flat surface approximately aligned with a vertical axis of the bioreactor 502. Further, in certain cases, the substrate 506 is both a metal substrate and includes a textured surface to increase adherence of a layer of the hydrogel 508 (e.g., a first layer of the hydrogel 508).

Additionally, or alternatively, in certain implementations within the bioreactor, a first layer of the hydrogel 508 adheres to the substrate 506 and is seeded with a first set of cells. Further, a second layer of the hydrogel 508 covers (in whole or in part) the first layer of the hydrogel 508 and is seeded with a second set of cells. The first set of cells and the second set of cells can likewise comprise different cell types. For instance, in some embodiments, the first set of cells comprises a first cell type of at least one of myogenic progenitors, adipogenic progenitors, or mesenchymal progenitors. Similarly, in some cases, the second set of cells comprises a second cell type (differing from the first cell type) of at least one of myogenic progenitors, adipogenic progenitors, or mesenchymal progenitors.

When applying pressure to cells in a sealed environment, in certain cases, the disclosed method also comprises pressurizing and depressurizing the hydrogel 508 and the cell mass 510 according to a growth-pressurization sequence. Accordingly, in some embodiments, the bioreactor 502 includes an inlet and an outlet whereby (and through which) the bioreactor 502 is pressurized. As illustrated in FIG. 5, a growth-pressurization sequence can include a growing time period 516, a pressurizing time period 518, and a depressurizing time period 520. The pressurizing time period 518 and the depressurizing time period 520 can accordingly be a first segment of time for pressurizing the cell mass 510 and a second segment of time for depressurizing the cell mass 510. During the growing time period 516, the seeded cells are grown into the cell mass 510. In some embodiments, the growing time period 516 comprises a determined amount of time (e.g., 3 days). In other embodiments, the growing time period 516 is determined based on the proportion of hydrogel 508 that has been consumed or repurposed by the cell mass 510. For example, the disclosed method can include determining the end of the growing time period 516 when 97% of the hydrogel 508 volume has been consumed or repurposed by the cell mass 510. During the growing time period 516, the seeded cells may be provided with a buffer or media solution that helps the cells degrade the hydrogel 508. For example, the buffer or media can include collagen enzymes to facilitate the degradation of the hydrogel 508.

After or during the growing time period 516, the disclosed method can apply pressure according to a pressurization schedule 526. The pressurization schedule 526 illustrated in FIG. 5 includes the pressurizing time period 518 and the depressurizing time period 520. Depending on the cell type and the stage of the culture process, cyclic strain can promote cell proliferation, differentiation, and hypertrophy. In some embodiments, and as illustrated in FIG. 5, the pressurization schedule 526 is a short strain schedule of pressurization for 1 hour per 24-hour period. Shorter strain schedules often result in increased cellular proliferation. Alternatively, the pressurization schedule 526 can comprise a longer strain schedule of pressurization for 18 hours per 24-hour period. Longer strain schedules can induce increased proliferation while also directing alignment of the cells within the cell mass 510. Strain cycles intermediate to these two ends of the spectrum are also contemplated here.

Pressurization schedules may also be shorter. To illustrate, a shorter pressurization schedule can include a pressurizing time period of 10 seconds and a depressurizing time period of 1 minute. The pressurization schedule can be performed once or repeated any number of times. Furthermore, different pressurization schedules may be combined, for example, pressurizing the bioreactor 502 for 1 hour, depressurizing for 23 hours, pressurizing for 18 hours, and depressurizing for 6 hours.

In some embodiments, the disclosed method comprises pressurizing the bioreactor 502 utilizing a pulsatile pressure. In particular, the disclosed method can include emitting pulses of pressure toward the cell mass within the sealed environment. During the pressurizing time period 518, in some cases, the disclosed method includes using different strain profiles at different frequencies. The peristaltic pump 524 (or other suitable pump) causes pressure variations or jolts to create these strain profiles. Different cell types often respond to different strain profiles. For example, myoblast proliferation is enhanced by cyclic strain of -10%-25% at a frequency of 0.5-2 Hz. To promote myoblast differentiation, strain profiles of ~2% at a frequency of -0.25 Hz have been found to promote upregulation of myogenic differentiation factors.

Strain profiles can be customized in conjunction with pressurization schedules to enhance proliferation, differentiation, and hypertrophy. To illustrate, in some cases, the disclosed method uses a cyclic strain schedule of -10% strain at a frequency of 0.25 Hz for ~8 hours per 24-hour period to induce fibroblast extracellular matrix production. As suggested above, the disclosed method can comprise combining different pressurization schedules with unique strain profiles to mechanically stretch the hydrogel 508 and the cell mass 510.

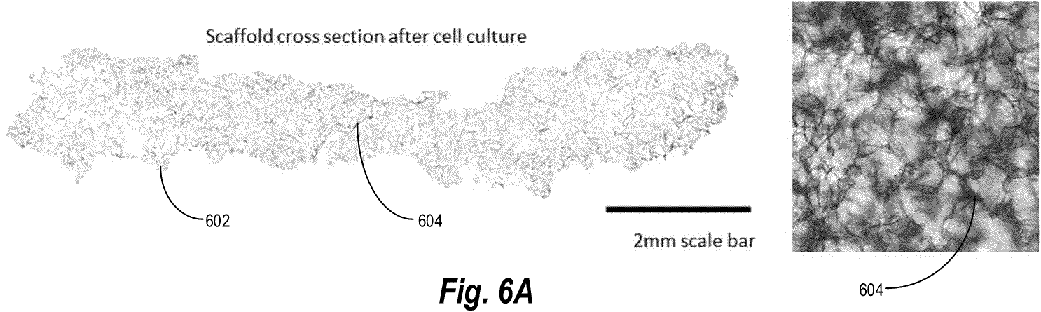

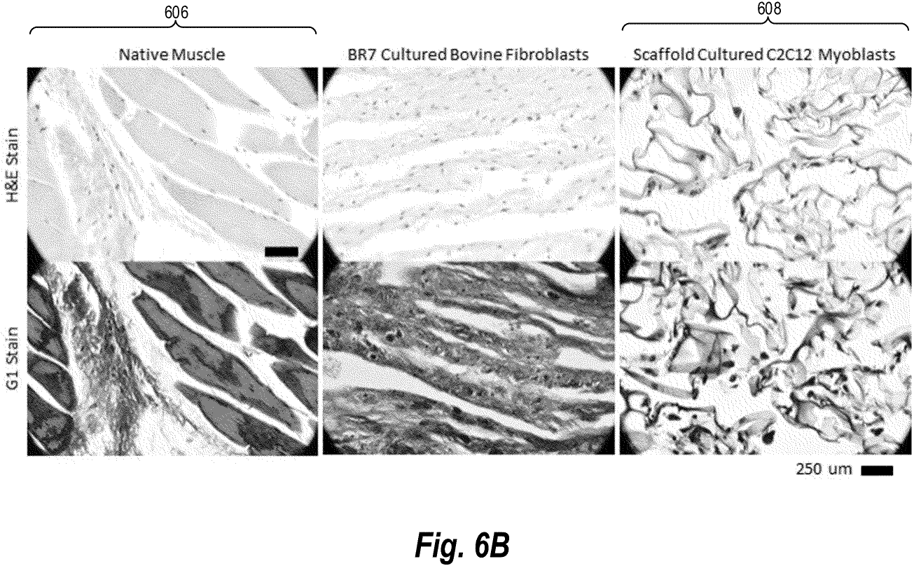

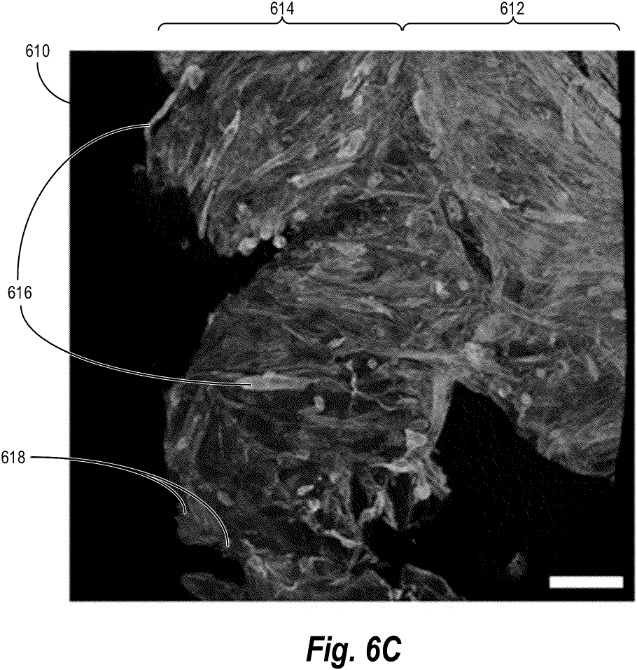

The foregoing discussion described various benefits of pressurizing a hydrogel and cell mass. FIGS. 6A-6C include various images that illustrate some benefits of growing cells within a hydrogel and inducing cells (or the cell-hydrogel matrix) to stretch in accordance with one or more embodiments. FIG. 6A illustrates a histological cross-section of a porous hydrogel showing uniform infiltration of the hydrogel scaffold by cells. Of particular note, this figure illustrates the formation of a robust 3-dimensional tissue that can be achieved using the techniques of the present disclosure. FIG. 6B illustrates various similarities between conventional meat cells and hydrogel-cultured cells in accordance with one or more embodiments. FIG. 6C illustrates how hydrogel scaffolds support cellular differentiation in accordance with one or more embodiments.

As mentioned, FIG. 6A illustrates a histological cross section of a hydrogel 602 after cell culture. Myoblasts were cultured in the hydrogel 602, which is a porous gelatin scaffold, in proliferating conditions for 5 days. After culture, cells 604 were stained (e.g., red) to demonstrate uniform infiltration of the hydrogel scaffold by cells 604. In particular, the channels and pores within the hydrogel 602 enable the cells 604 to penetrate deeply into the hydrogel 602. Furthermore, the hydrogel 602 provides nutrition to the cells 604 and enables 3-dimensional proliferation of the cells 604. In particular, the hydrogel 602 provides additional surface area to which the cells 604 attach and uptake proteins, micronutrients, lipids, and other materials from the hydrogel 602. Thus, the cells 604 are able to grow in a 3-dimensional structure where cells in the center of the hydrogel 602 are still able to access nutrition to grow.

The hydrogel 602 also provides physical support for the cells 604. In particular, the hydrogel 602 provides an elastic modulus favorable for cell growth. The hydrogel 602 provides means by which the cells 604 may be mechanically stretched under pressurization. In particular, the hydrogel 602 provides room and support for the cells 604 to be stretched. The hydrogel 602 distributes pressure and supports the cells 604 as they are mechanically stretched to facilitate hypertrophy.

FIG. 6B illustrates histological cross-sections of conventional muscle 606 compared with hydrogel cultured myoblasts 608. In particular, FIG. 6B illustrates how the stretched hydrogel-cell matrix results in cell alignment and robust muscle fiber alignment. For example, the hydrogel cultured myoblasts 608 demonstrate larger bulkier fibers that more-closely mimic the conventional muscle 606 than cells that have not been stretched. Furthermore, as illustrated in FIG. 6B, the hydrogel cultured myoblasts 608 demonstrate a more amorphous structure than the more crystalline-like structure shown by the conventional muscle 606 or in the BR7 cultured bovine fibroblasts, which have been grown in a roller bottle. Despite this difference, hydrogel can be further processed to better mimic the conventional muscle 606. For example, in some embodiments, the hydrogel can be dehydrated to toughen the hydrogel. The hydrogel may also comprise a high-protein hydrogel that increases the protein content of the finished cell-based product.

FIG. 6C illustrates a histological cross-section demonstrating how the hydrogel supports cellular differentiation. In particular, the cross-section 610 portrays myoblasts that were cultured on a porous hydrogel scaffold in proliferating conditions for 3 days followed by differentiation conditions for 2 days. The hydrogel scaffold was cut with a razor, stained, and the cross-sectional edge was imaged. The cross-section 610 includes an image of a superficial face 612 of a cell mass and a cross-section 614 of the cell mass.

As illustrated in FIG. 6C, the superficial face 612 demonstrates a dense concentration of cells. Furthermore, the cross-section 610 shows a high density of cell nuclei (including the nuclei 618). The high cell density demonstrates improved cell growth. The cross-section 614 of the cell mass shows a network of cells throughout the hydrogel at various stages of maturation. The stain of the cells 616 indicates that the cells 616 are heavy in myosin, which is found in mature muscle. Other cells within the cross-section 610 are at different stages of maturation.

More specifically, and as illustrated in FIG. 6C, the hydrogel and the cells may be stained to highlight cells at different stages of maturation. As illustrated in FIG. 6C, a phalloidin stain (e.g., green) of F-actin filaments in the cytoskeleton stains a structural protein ubiquitous to the cells. Phalloidin stain is seen throughout the cross-section 610 and is more prevalent on the superficial face 612 of the cell mass. 4′, 6-diamidino-2-phenylindole (DAPI) stain (e.g., blue) indicates the location of the nuclei 618. Myosin heavy chain stain (e.g., red) can label protein found in the cells 616.

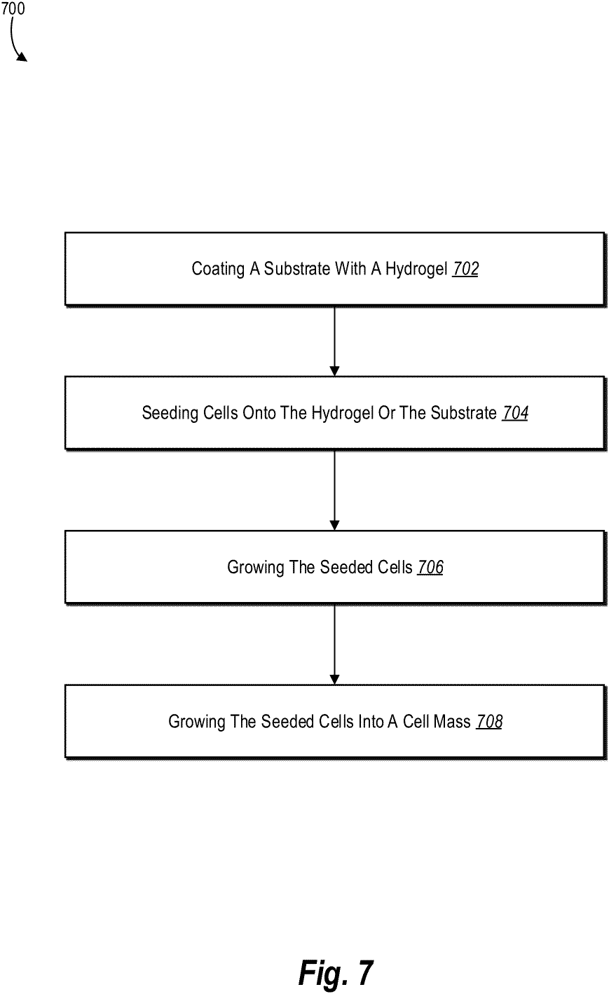

FIGS. 1A-6C, the corresponding text, and the examples provide several different systems, methods, techniques, components, and/or devices relating to the pressurizing a cell-hydrogel matrix in accordance with one or more embodiments. In addition to the above description, one or more embodiments can also be described in terms of flowcharts including acts for accomplishing a particular result. FIG. 7 illustrates such a flowchart of acts. Additionally, the acts described herein may be repeated or performed in parallel with one another or in parallel with different instances of the same or similar acts.

FIG. 7 illustrates a flowchart of a series of acts 700. By way of overview, the series of acts 700 includes an act 702 of coating a substrate with a hydrogel, an act 704 of seeding cells onto the hydrogel or the substrate, an act 706 of growing the seeded cells into a cell mass, and an act 708 of pressurizing the cell mass.

As illustrated in FIG. 7, the series of acts 700 includes the act 702 of coating a substrate with a hydrogel. In particular, in some embodiments, the act 702 comprises coating a substrate within a bioreactor with hydrogel. In some embodiments, the act 702 further comprises flooding the bioreactor containing the substrate with a hydrogel solution, draining the bioreactor of the hydrogel solution, and gelating the hydrogel solution to form the hydrogel. In some embodiments, coating the substrate with the hydrogel comprises spray coating the substrate utilizing one or more nozzles.

The series of acts 700 further includes the act 704 of seeding cells onto the hydrogel or the substrate. In particular, the act 704 comprises seeding cells onto at least one of the hydrogel or the substrate. In some embodiments, the act 704 further comprises incorporating the cells into a hydrogel solution, distributing the hydrogel solution onto the substrate, and gelating the hydrogel solution to form the hydrogel.

In some embodiments, the acts 702 and 704 further comprise incorporating a first cell type into a first hydrogel solution to create a first blend of hydrogel and cells, adding a layer of the first blend over the substrate, incorporating a second cell type into a second hydrogel solution to create a second blend of hydrogel and cells, and adding a layer of the second blend over the layer of the first blend. Furthermore, in yet other embodiments, the acts 702 and 704 comprise adding a first layer of hydrogel over the substrate, adding cells to the first layer of hydrogel with a first set of cells, adding a second layer of hydrogel over the first layer of hydrogel, and seeding the second layer of hydrogel with a second set of cells.

FIG. 7 further illustrates the act 706 of growing the seeded cells into a cell mass. In some embodiments, the act 706 comprises growing the seeded cells into a cell mass for a growing time period.

The series of acts 700 also includes the act 708 of pressurizing the cell mass. In particular, the act 708 comprises pressurizing the cell mass and the hydrogel within a sealed environment to induce the seeded cells to stretch. In some embodiments, the act 708 further comprises emitting pulses of pressure toward the cell mass within the sealed environment. Additionally, in some embodiments, the act 708 comprises creating a pressure gradient across a height of the hydrogel and a pressure gradient across a length of the hydrogel. Additionally, in some embodiments, the act 708 comprises pressurizing the cell mass and the hydrogel within the bioreactor for a pressurizing time period to induce the seeded cells to stretch. In some embodiments, pressurizing the cell mass comprises utilizing a peristaltic pump to emit pulses of pressure for the pressurizing time period.

In some embodiments, the act 708 of pressurizing the cell mass further comprises determining that the cells are in a differentiation stage, pressurizing the cell mass for a first segment of time, depressurizing the cell mass for a second segment of time, and repeating one or more cycles of pressurizing and depressurizing the cell mass. In some embodiments, the first segment of time is ten seconds, and the second segment of time is one minute.

The series of acts 700 can further comprise an additional act of depressurizing the cell mass for a depressurizing time period. The series of acts 700 may further comprise repeating one or more cycles of pressurizing and depressurizing the cell mass.

The series of acts 700 can further comprise an additional act of creating pores within the hydrogel by freeze drying the hydrogel, and wherein seeding the cells onto the hydrogel comprises circulating the cells through the pores within the hydrogel.

The series of acts 700 can further comprise an additional act of separating the cell mass and the hydrogel from the substrate, solubilizing the hydrogel by heating the hydrogel, and separating the cell mass and the solubilized hydrogel by utilizing at least one of centrifugation, filtration, or settling.

If the hydrogel is comprised of edible material, it may become part of the final product. The series of acts 700 can further comprise of retaining the cell mass within the hydrogel and separating this cell mass and hydrogel mixture from the substrate. The hydrogel may add beneficial organoleptic and texture to the product that make the tasting experience more similar to that of slaughtered meat.

As described, the disclosed method can comprise various steps for stretching hydrogel and cells to induce hypertrophy. In some embodiments, regardless of methods described above, this disclosure includes a bioreactor for growing a cell mass comprising: a metal substrate; a first layer of hydrogel that adheres to the metal substrate and is seeded with a first set of cells; and a second layer of hydrogel that covers in whole or in part the first layer of hydrogel and is seeded with a second set of cells. As indicated above, in certain implementations, the bioreactor further comprises an inlet and an outlet whereby the bioreactor may be pressurized.

In some embodiments, the metal substrate comprises a metal sheet with a flat surface approximately aligned with a vertical axis of the bioreactor. Additionally or alternatively, in one or more implementations, the metal substrate is textured to increase adherence of the first layer of hydrogel. Further, in certain embodiments, the first set of cells comprises a first cell type and the second set of cells comprises a second cell type, wherein the first cell type and the second cell type comprise at least one of myogenic progenitors, adipogenic progenitors, or mesenchymal progenitors.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A method for inducing cells within a growing cell mass to stretch, the method comprising:
coating a substrate with hydrogel;
seeding cells onto at least one of the hydrogel or the substrate;
growing the seeded cells into a cell mass; and
pressurizing the cell mass and the hydrogel within a sealed environment to induce the seeded cells to stretch.

2. The method of claim 1, wherein coating the substrate and seeding the cells comprises:
incorporating a first cell type into a first hydrogel solution to create a first blend of hydrogel and cells;
adding a layer of the first blend over the substrate;
incorporating a second cell type into a second hydrogel solution to create a second blend of hydrogel and cells; and
adding a layer of the second blend over the layer of the first blend.

3. The method of claim 1, wherein pressurizing the cell mass comprises emitting pulses of pressure toward the cell mass within the sealed environment.

4. The method of claim 1, wherein pressurizing the cell mass and the hydrogel within the sealed environment comprises creating a pressure gradient across a height of the hydrogel and a pressure gradient across a length of the hydrogel.

5. The method of claim 1, wherein seeding the cells onto the hydrogel comprises:
incorporating the cells into a hydrogel solution;
distributing the hydrogel solution onto the substrate; and
gelating the hydrogel solution to form the hydrogel.

6. The method of claim 1, wherein pressurizing the cell mass comprises:
determining that the cells are in a differentiation stage;
pressurizing the cell mass for a first segment of time;
depressurizing the cell mass for a second segment of time; and
repeating one or more cycles of pressurizing and depressurizing the cell mass.

7. The method of claim 6, wherein:
the first segment of time is ten seconds; and
the second segment of time is one minute.

8. The method of claim 1, further comprising:
creating pores within the hydrogel by freeze drying the hydrogel; and
wherein seeding the cells onto the hydrogel comprises circulating the cells through the pores within the hydrogel.

9. The method of claim 1, wherein coating the substrate and seeding the cells comprises:
adding a first layer of hydrogel over the substrate;
adding cells to the first layer of hydrogel with a first set of cells;
adding a second layer of hydrogel over the first layer of hydrogel; and
seeding the second layer of hydrogel with a second set of cells.

10. A method for inducing cells within a growing cell mass to stretch, the method comprising:
coating a substrate within a bioreactor with hydrogel;
seeding cells onto at least one of the hydrogel or the substrate;
growing the seeded cells into a cell mass for a growing time period;
pressurizing the cell mass and the hydrogel within the bioreactor for a pressurizing time period to induce the seeded cells to stretch; and
depressurizing the cell mass for a depressurizing time period.

11. The method of claim 10, further comprising repeating one or more cycles of pressurizing and depressurizing the cell mass.

12. The method of claim 11, wherein coating the substrate with the hydrogel comprises:
flooding the bioreactor containing the substrate with a hydrogel solution;
draining the bioreactor of the hydrogel solution; and
gelating the hydrogel solution to form the hydrogel.

13. The method of claim 11, wherein coating the substrate with the hydrogel comprises spray coating the substrate utilizing one or more nozzles.

14. The method of claim 11, wherein pressurizing the cell mass comprises utilizing a peristaltic pump to emit pulses of pressure for the pressurizing time period.

15. The method of claim 11, further comprising:
separating the cell mass and the hydrogel from the substrate;
solubilizing the hydrogel by heating the hydrogel; and
separating the cell mass and the solubilized hydrogel by utilizing at least one of centrifugation, filtration, or settling.

16. A bioreactor for growing a cell mass, the bioreactor comprising:
a metal substrate;
a first layer of hydrogel that adheres to the metal substrate and is seeded with a first set of cells; and
a second layer of hydrogel that covers in whole or in part the first layer of hydrogel and is seeded with a second set of cells.

17. The bioreactor of claim 16, wherein the metal substrate comprises a metal sheet with a flat surface approximately aligned with a vertical axis of the bioreactor.

18. The bioreactor of claim 16, wherein the first set of cells comprises a first cell type and the second set of cells comprises a second cell type, wherein the first cell type and the second cell type comprise at least one of myogenic progenitors, adipogenic progenitors, or mesenchymal progenitors.

19. The bioreactor of claim 16 further comprising an inlet and an outlet whereby the bioreactor may be pressurized.

20. The bioreactor of claim 16, wherein the metal substrate is textured to increase adherence of the first layer of hydrogel.
